(19) United States
(12) Patent Application Publication
(10) Pub. No.: US 2026/0245967 A1
(43) Pub. Date: Aug. 20, 2026

(54) SODIUM SALTS HAVING BORATE OR ALUMINATE ANIONS

(71) Applicant: Sumitomo Chemical Co., Ltd, Tokyo (JP)

(72) Inventor: Florence Bourcet, Godmanchester (GB)

(73) Assignee: Sumitomo Chemical Co., Ltd, Tokyo (JP)

(21) Appl. No.: 19/470,875

(22) PCT Filed: Mar. 20, 2024
(86) PCT No.: PCT/EP2024/057489

§ 371 Date: Sep. 30, 2025

(30) Foreign Application Priority Data
Mar. 30, 2023 (GB) 2304745.9

(51) Int. Cl. H01M 10/0568; C07F 5/02; H01M 10/054; H01M 10/0569

(52) U.S. Cl. CPC H01M 10/0568; C07F 5/022; H01M 10/054; H01M 10/0569; H01M 2300/0028

(57) ABSTRACT

A compound of formula (I) or (II): (I) (II) X is Al or B; R¹ in each occurrence is independently a substituent and two R¹ groups may be linked to form a ring, with the proviso that at least one R¹ is a partially fluorinated linear or branched C₁₋₂₀ alkyl group in which one or more non-adjacent, non-terminal C atoms other than the C atom bound to O of X—O may be replaced with O and wherein the fluorinated C₁₋₂₀ alkyl group includes at least one group of formula —CF₂H or —CFH₂; and R² in each occurrence is independently a divalent organic group The compounds of formula (I) or (II) may be used in the electrolyte of a sodium battery or sodium-ion battery.

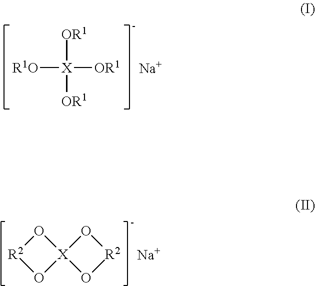

### BACKGROUND

Lithium batteries and lithium-ion batteries containing a lithium salt have been the subject of extensive investigation. However, the scarcity of lithium is a drawback. Consequently, batteries containing more abundant metals such as sodium, magnesium and potassium have been investigated.

D. Ould et al, “Sodium Borates: Expanding the Electrolyte Selection for Sodium-Ion Batteries”, Angew. Chem. Int. Ed. 2022, 61, e202202133 discloses alkoxyborate sodium salts for sodium-ion batteries. WO2021/138370 discloses lithium metal batteries having an ionic liquid electrolyte with a sodium additive.

EP3783720 discloses an electrolyte solution comprising a sodium or potassium bis(oxalato) borate salt and an organic solvent comprising a pyrrolidone and/or a phosphoric acid compound.

US 2021/036358 discloses an SO₂-based electrolyte for a rechargeable battery cell containing a conducting salt of Formula (I), in which M is selected from alkali metals, alkaline earth metals, metals of group 12 and aluminium:

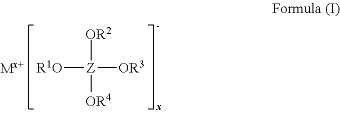

US2018/019498 discloses a nonaqueous electrolytic solution for a secondary battery including at least one of boric acid esters, acid anhydrides, cyclic carbonates having an unsaturated bod, cyclic carbonates having a halogen atom, cyclic sulfonic acid esters and amines having an acetoacetyl group.

### SUMMARY

The present disclosure provides a compound of formula (II):

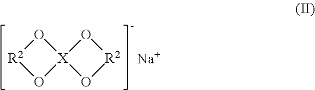

wherein X is Al or B and R² in each occurrence is independently a divalent organic group selected from:
unsubstituted or substituted C₆₋₂₀ arylene or C₅₋₂₀ heteroarylene;
unsubstituted or substituted C₁₋₂₀ alkylene wherein one or more non-adjacent C atoms other than the C atoms bound to O of the borate may be replaced with O and one or more H atoms may be replaced with F; and
C₁₋₂₀-alkylene-C₆₋₂₀ arylene or C₁₋₂₀-alkylene-C₅₋₂₀ heteroarylene wherein one or more non-adjacent C atoms of the C₁₋₂₀ alkylene other than the C atoms bound to O of the borate may be replaced with O, the C₁₋₂₀-alkylene is unsubstituted or substituted; and the C₆₋₂₀ arylene is substituted or unsubstituted.

Optionally, at least one R² is a group of formula (III):

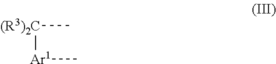

wherein R³ in each occurrence is H or a substituent and Ar¹ is an unsubstituted or substituted C₆₋₂₀ arylene or C₅₋₂₀ heteroarylene group.

Optionally, Ar¹ is unsubstituted or substituted 1,2-phenylene.

Optionally, each R² is a group of formula (III).

The present disclosure provides a compound of formula (I):

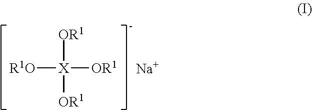

wherein X is Al or B; and
R¹ in each occurrence is independently a substituent and two R¹ groups may be linked to form a ring, with the proviso that at least one R¹ is a partially fluorinated linear or branched C₁₋₂₀ alkyl group in which one or more non-adjacent, non-terminal C atoms other than the C atom bound to O of X—O may be replaced with O and wherein the fluorinated C₁₋₂₀ alkyl group includes at least one group of formula —CF₂H or —CFH₂.

Optionally, each R¹ is a partially fluorinated linear or branched C₁₋₂₀ alkyl group in which one or more non-adjacent, non-terminal C atoms other than the C atom bound to O of X—O may be replaced with O and wherein the fluorinated C₁₋₂₀ alkyl group includes at least one group of formula —CF₂H or —CFH₂.

The present disclosure provides an electrolyte comprising a compound of formula (I) or (II) and one or more solvents.

Optionally, the one or more solvents include at least one of C₂₋₁₀ alkylene carbonates and di(C₁₋₁₀ alkyl) carbonates.

Optionally, the electrolyte contains at least one mole of sodium cations per 10 moles of solvent.

Optionally, the concentration of the compound of formula (I) or (II) is at least 0.25 moles/litre.

The present disclosure provides a battery comprising an anode, a cathode and an electrolyte comprising a compound of formula (I) or (II) disposed between the anode and the cathode.

Optionally, the battery contains no more than 10 moles of total solvent per mole of Na+.

### DESCRIPTION OF DRAWINGS

FIG. 1 is a schematic illustration of a battery containing a compound according to embodiments of the present disclosure;

FIG. 2 shows the circuit used to determine impedance of an electrolyte;

FIG. 3A shows Nyquist plots for cells containing Compound Examples 1 and 2 according to some embodiments of the present disclosure; and

FIG. 3B shows magnification of the Nyquist plots of FIG. 3A.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to an element of the Periodic Table include any isotopes of that element.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

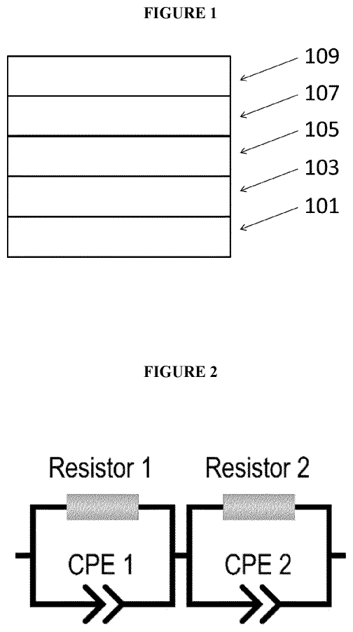

FIG. 1 illustrates a battery. The battery may be a sodium battery or a sodium ion battery. The battery is suitably a secondary (rechargeable) battery.

The battery comprises an anode current collector 101 in contact with an anode 103 on a surface thereof; a cathode current collector 109 in contact with a cathode 107; and a layer 105 comprising or consisting of an electrolyte as described herein disposed between the anode and cathode.

In some embodiments, layer 105 is a gel layer comprising a polymer; a solvent; and a compound of formula (I).

In some embodiments, layer 105 is a porous separator comprising an electrolyte as described herein absorbed into the porous separator.

In the case of a sodium ion battery, the anode comprises an active material, e.g., graphite, for absorption of the metal ions.

In the case of a sodium battery, the anode 103 is a layer of sodium which is formed over the anode current collector during charging of the battery and which is stripped during discharge of the battery.

The cathode may be selected from any cathode known to the skilled person.

The anode and cathode current collectors may be any suitable conductive material known to the skilled person, e.g. one or more layers of metal or metal alloy such as aluminium or copper.

For simplicity, FIG. 1 illustrates a battery in which the anode and cathode are separated only by a layer comprising or consisting of the electrolyte, however it will be understood that in use a solid-electrolyte interphase will typically form on the anode surface.

In other embodiments, one or more further layers may be disposed between the anode and the electrolyte and/or the cathode and the electrolyte.

A battery as described herein or a battery pack comprising a plurality of batteries as described herein may be used, without limitation, to power electric vehicles or stationary objects.

### Compounds of Formula (I)

Formula (I) is:

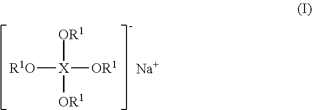

X is Al or B.
R¹ in each occurrence is independently a substituent wherein two R¹ groups may be linked to form a ring.

Preferably, each R¹ is independently selected from:
linear, branched or cyclic C₁₋₂₀ alkyl wherein one or more non-adjacent, non-terminal C atoms other than the C atom bound to O of X—O may be replaced with O and one or more H atoms may be replaced with F;
an optionally substituted C₆₋₂₀ aryl or C₅₋₂₀ heteroaryl; and
C₁₋₂₀ alkylene-C₆₋₂₀ aryl or C₁₋₂₀ alkylene-C₅₋₂₀ heteroaryl wherein one or more non-adjacent, non-terminal C atoms of the C₁₋₂₀ alkylene other than the C atom bound to O of the borate may be replaced with O and one or more H atoms of the C₁₋₂₀ alkylene may be replaced with F, and the C₆₋₂₀ aryl or C₅₋₂₀ heteroaryl may be unsubstituted or substituted.

Where present, substituents of the C₆₋₂₀ aryl or C₅₋₂₀ heteroaryl are preferably and independently selected from substituents R⁵ wherein R⁵ in each occurrence is independently selected from the group consisting of F and C₁₋₁₂ alkyl wherein one or more non-adjacent, non-terminal C atoms of the C₁₋₁₂ alkyl may be replaced with O, S, NR⁴, CO COO or CONR⁴ wherein R⁴ in each occurrence is independently a C₁₋₁₂ hydrocarbyl group, and one or more H atoms of the C₁₋₁₂ alkyl group may be replaced with F.

By “non-terminal C atom” of an alkyl chain as used herein is meant the methyl group at the chain end of a linear alkyl chain or each one of the methyl groups at the chain ends of a branched alkyl group.

A C₁₋₁₂ hydrocarbyl group as described anywhere herein is preferably selected from C₁₋₁₂ alkyl; phenyl; and phenyl substituted with one or more C₁₋₆ alkyl groups.

In the case where at least two R¹ groups are not linked, preferably each R¹ independently in each occurrence is a partially fluorinated linear or branched C₁₋₂₀ alkyl group in which one or more non-adjacent, non-terminal C atoms other than the C atom bound to O of X—O may be replaced with O and wherein the fluorinated C₁₋₂₀ alkyl group includes at least one group of formula —CF₂H or —CFH₂.

In some embodiments, two pairs of R¹ groups are linked and the compound of formula (I) has formula (II):

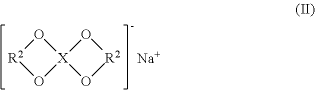

R² in each occurrence is independently a divalent organic group.

Preferably, R² is selected from:
unsubstituted or substituted C₆₋₂₀ arylene or C₅₋₂₀ heteroarylene;
unsubstituted or substituted C₁₋₂₀ alkylene wherein one or more non-adjacent C atoms other than the C atoms bound to O of the borate may be replaced with O and one or more H atoms may be replaced with F; and
C₁₋₂₀-alkylene-C₆₋₂₀ arylene or C₁₋₂₀-alkylene-C₅₋₂₀ heteroarylene wherein one or more non-adjacent C atoms of the C₁₋₂₀ alkylene other than the C atoms bound to O of the borate may be replaced with O, the C₁₋₂₀-alkylene is unsubstituted or substituted; and the C₆₋₂₀ arylene or C₅₋₂₀ heteroarylene is substituted or unsubstituted.

The C₆₋₂₀ arylene or C₅₋₂₀ heteroarylene may be unsubstituted or substituted with one or more substituents. Preferred substituents, where present, are selected from R⁵ as described above.

R² is preferably selected from: Ar¹ wherein Ar¹ in each occurrence is independent an optionally substituted C₆₋₂₀ arylene or C₅₋₂₀ heteroarylene group, e.g., 1,2-phenylene, which may be unsubstituted or substituted with one or more substituents; a bi-arylene group of formula Ar¹—Ar¹, for example 2,2′-linked biphenylene which may be unsubstituted or substituted with one or more substituents; ethylene; propylene; and a group of formula (III):

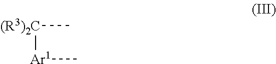

wherein R³ in each occurrence is H, F or a C₁₋₆ alkyl in which one or more H atoms may be replaced with F; and Ar¹ is a C₆₋₂₀ arylene group, preferably unsubstituted or substituted 1,2-phenylene.

In a preferred embodiment, at least one R³, optionally each R³, is a C₁₋₆ perfluoroalkyl group.

Preferably, each Ar¹ of formula (I) is phenylene, more preferably a 1,2-linked phenylene which is unsubstituted or substituted with one or more substituents.

Where present, substituents of Ar¹ are preferably and independently selected from R⁵ as described above

The compound of formula (I) or (II) may be formed by reacting a compound of formula (IV) with either a compound of formula (Va) or (Vb) in the case where R¹ groups are not linked, or a compound of formula (VIa), (VIb) or (Vic) in the case of compounds of formula (II):

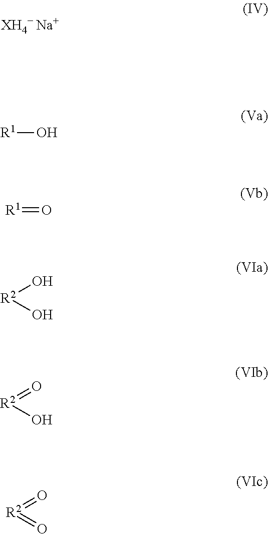

The compound of formula (Va) may be a primary, secondary or tertiary alcohol.

The compound of formula (Vb) may be an aldehyde or a ketone.

Exemplary compounds of formula (IV) include, without limitation, sodium aluminium hydride (NaAlH₄) and sodium borohydride (NaBH₄).

### Solvent

An electrolyte containing one or more solvents and a compound of formula (I) or (II) may include one or more solvents selected from C₂₋₁₀ alkylene carbonates, di(C₁₋₁₀ alkyl) carbonates, for example propylene carbonate, ethylene carbonate, dimethyl carbonate, diethyl carbonate and fluorinated analogues thereof; linear, branched or cyclic compounds containing two or more ether groups, for example 1,3-dioxolane, 2,5dimethoxy tetrahydrofuran, glyme (dimethoxyethane), diglyme, triglyme and tetraglyme; diethylene glycol diethyl ether; succinonitrile; cyclic lactones and mixtures thereof.

Optionally, a ratio of moles of solvent:moles of Na+ of the electrolyte is no more than 20:1, optionally less than or equal to 15:1 or less than or equal to 10:1.

### Polymer

An electrolyte as described herein may comprise a polymer, in which case the electrolyte may be a gel.

The polymer may be selected from any known ion-conducting polymers including, without limitation: poly(alkylene oxide), for example poly(ethylene oxide) and poly(propylene oxide); and fluorinated polymers such as PVDF, PVDF-HFP; PMMA; polyacrylonitrile; polycarbonate; polyethylene; polypropylene; poly(vinyl methyl ketone); polyvinylpyrrolidone; polyether ether ketone; polyisoprene; polybutadiene; polystyrene-block-polyisoprene-block-polystyrene; poly(1-vinylpyrrolidone-co-vinyl acetate); polystyrene-block-polybutadiene-block-polystyrene; polystyrene-block-poly(ethylene oxide)-block-polystyrene; and copolymers and mixtures thereof.

The polymer is suitably a neutral polymer, i.e., not a polymer substituted with ionic groups, and in particular is suitably not a single-ion conducting polymer comprising anionic groups.

### EXAMPLES

### Compound Example 1

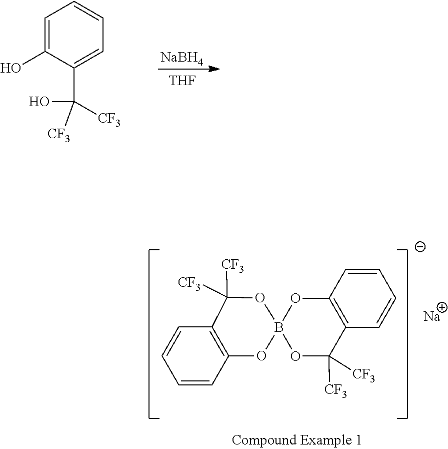

The reaction was performed under nitrogen atmosphere. A solution of NaBH₄ (151 mg, 4 mmol, 0.34 M) in anhydrous tetrahydrofuran was added dropwise to a solution of (1,1,1,3,3,3-hexafluoro-2-hydroxypropan-2-yl) phenol (2.08 g, 8 mmol) in 12 mL of tetrahydrofuran at room temperature. The mixture was then stirred at room temperature for 3 hours and at 65° C. for 3 hours. Additional solution of NaBH₄ (55 mg, 1.45 mmol, 0.72 M) in THF was added dropwise. Then the reaction mixture was stirred overnight at room temperature. After reaction completion, propylene carbonate (1.4 mL, 16 mmol) was added. The excess solvent was removed under reduced pressure at 30° C. (3.3×10⁻² mbar) for 3 hours to yield a thick transparent oil.

¹H NMR (600 MHz) in THF-d8: δ (ppm), 1.37 (d, CH₃, from propylene carbonate 20.7H), 3.96 (m, CH, from propylene carbonate 6.7H), 4.50 (t, CH, from propylene carbonate 6.7H), 4.80 (m, CH, from propylene carbonate 6.1H), 6.73 (m, 4H), 7.16 (td, J=7.6 Hz, J=1.7 Hz, 2H), 7.33 (d, J=7.8 Hz, 2H).

From integration of NMR peaks, it was calculated that for two molecules of (1,1,1,3,3,3-hexafluoro-2-hydroxypropan-2-yl) phenol, which correspond to one sodium cation, 6.70 molecules of propylene carbonate per sodium cation was present as residual solvent.

### Compound Example 2

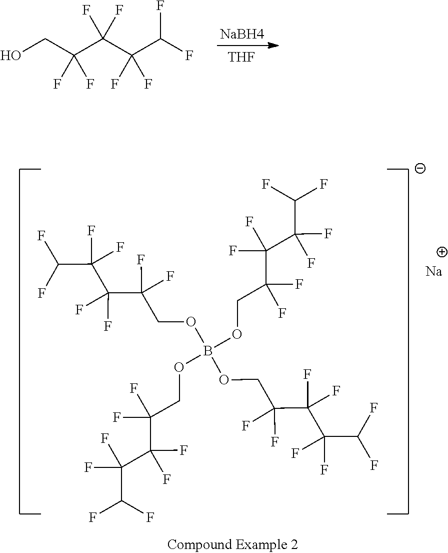

The reaction was performed under nitrogen atmosphere. A solution of NaBH₄ (203 mg, 5.37 mmol, 0.45 M) in anhydrous tetrahydrofuran was added dropwise to a solution of 2,2,3,3,4,4,5,5-octafluoropentan-1-ol (OFP) (3 mL, 21.5 mmol) in 12 mL of tetrahydrofuran at room temperature. The mixture was stirred at room temperature for 1 hour and at 65° C. for 4 hours. The reaction mixture was further stirred overnight at room temperature. After the reaction completion, propylene carbonate (0.68 mL, 8.06 mmol) was added. The excess solvent was then removed under reduced pressure at 30° C. (3.3×10⁻² mbar) for 3 hours to yield a thick transparent oil. Additional propylene carbonate was added to obtain a stable transparent liquid.

¹H NMR (600 MHz) in THF-d8: δ (ppm), 1.39 (d, CH₃, from propylene carbonate 14.7H), 3.90 (t, J=15.01 Hz, 7.7H), 3.97 (m, CH, from propylene carbonate 7.8H), 4.50 (t, CH, from propylene carbonate 4.7H), 4.80 (m, CH, from propylene carbonate 4.2H), 6.62 (tt, J=51.4 Hz, J=5.8 Hz, 4H).

From integration of NMR peaks, it was calculated that for four molecules of octafluoropentyloxy ligands in the product, which correspond to one sodium cation, 4.80 molecules of propylene carbonate per sodium cation was present as residual solvent.

### Cell Formation

2032-type coin cells were prepared cells fabricated in a rigorously dry and oxygen-free Argon gas-filled MBraun glovebox using casings purchased from Cambridge Energy Solutions.

A stainless-steel spacer was inserted in a coin cell bottom, followed by a fluoro-silicone stencil (purchased from Silex Silicones). The stencil was shaped as a disk of 15.5 mm diameter, with a circular hole of 5 mm diameter cut in its middle (the thickness of the stencil in the crimped cell was 360 μm). The hole was filled with 30 μl of an electrolyte formulation. On top of the stencil was placed a stainless-steel spacer, a wave spring and a coin cell top. Finally, the coin cell was crimped.

Electrolyte formulations used to form the coin cells contained either Compound Example 1, Compound Example 2, Comparative Compound 1 or Comparative Compound 2.

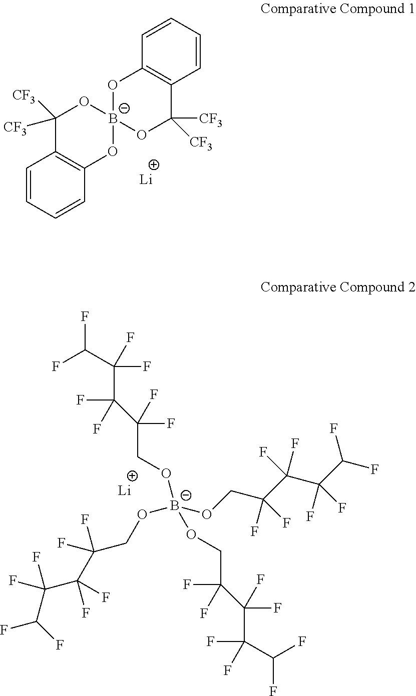 [SMILES: FC(F)(F)C1(C(F)(F)F)O[B-]2(Oc3ccccc31)Oc1ccccc1C(C(F)(F)F)(C(F)(F)F)O2 ; [Li+] ; FC(F)C(F)(F)C(F)(F)C(F)(F)CO[B-](OCC(F)(F)C(F)(F)C(F)(F)C(F)F)(OCC(F)(F)C(F)(F)C(F)(F)C(F)F)OCC(F)(F)C(F)(F)C(F)(F)C(F)F ; [Li+]]

### Electrochemical Impedance Spectroscopy (EIS)

EIS measurements of coin cells were conducted at room temperature using a potentiostat (Interface 1010E, Gamry Instruments).

The EIS spectrums were taken over a frequency range of 1 Hz to 1 MHz, with an amplitude of 5 mV.

Ionic conductivities were calculated using the following formula:

$$σ = \frac{l}{A \cdot R}$$

where:
l—thickness of the material between the two stainless disks, which corresponds to the thickness of the separator,
A—area of the hole in the separator,
R—impedance of the electrolyte.

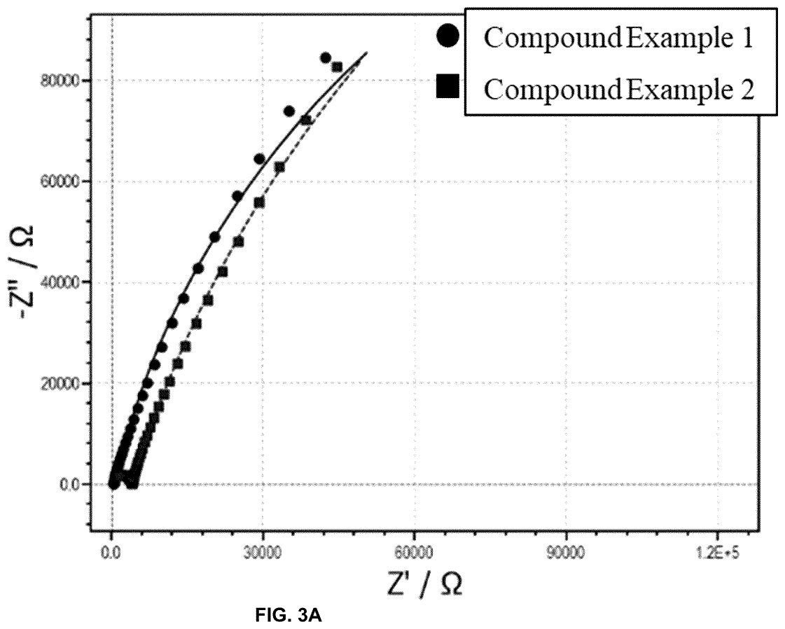

To determine the impedance, the Nyquist plot of each cell is fit with the equivalent circuit shown in FIG. 2, with the impedance of Resistor 1 being taken as the ionic resistance of the electrolyte in the system.

Resistor 1 corresponds to the x-intercept of the 1^{st} semi-circle on the x-axis of the Nyquist plot.

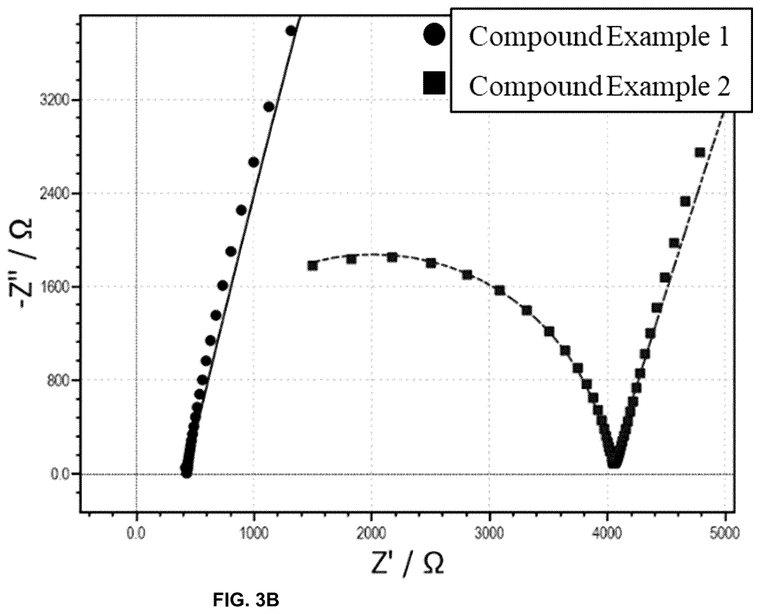

The Nyquist plots of Compound Examples 1 and 2 are shown in FIG. 3A. Magnified plots are shown in FIG. 3B.

Ionic conductivities are set out in Table 1.

TABLE 1

| | Propylene carbonate | Ionic Conductivity |
|---|---|---|
| Electrolyte | molecules per cation | S cm⁻¹ |
| Compound Example 1 | 6.7 | 4.4 × 10⁻⁴ |
| Compound Example 2 | 4.8 | 4.7 × 10⁻⁵ |
| Comparative Compound 1 | 6.6 | 2.8 × 10⁻⁴ |
| Comparative Compound 2 | 4.3 | 4.6 × 10⁻⁵ |

As set out in Table 1, surprisingly Compound Example has significantly higher ionic conductivity than Comparative Compound 1 and Compound Example 2 has similar ionic conductivity to Comparative Compound 2 despite the significantly larger size of the sodium cation of Compound Examples 1 and 2 as compared to the lithium cation of Comparative Compounds 1 and 2.

Further, the present inventors have surprisingly found that Compound Example 1 can be dissolved in propylene carbonate to give high concentration solutions, whereas D. Ould et al (ibid) discloses that Na[B(OCH₂(CF₂)₂CF₃)₄] has poor solubility in a 1:1 mixture of ethylene carbonate and diethylcarbonate.

1. A compound of formula (II): 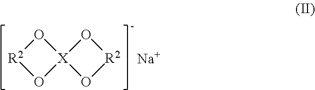
wherein X is Al or B and R² in each occurrence is independently a divalent organic group selected from:
unsubstituted or substituted C₆₋₂₀ arylene or C₅₋₂₀ heteroarylene;
unsubstituted or substituted C₁₋₂₀ alkylene wherein one or more non-adjacent C atoms other than the C atoms bound to O of the borate may be replaced with O and one or more H atoms may be replaced with F; and
C₁₋₂₀-alkylene-C₆₋₂₀ arylene or C₁₋₂₀-alkylene-C₅₋₂₀ heteroarylene wherein one or more non-adjacent C atoms of the C₁₋₂₀ alkylene other than the C atoms bound to O of the borate may be replaced with O, the C₁₋₂₀-alkylene is unsubstituted or substituted; and the C₆₋₂₀ arylene is substituted or unsubstituted.

2. The compound according to claim 1 wherein at least one R² is a group of formula (III): 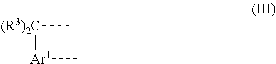
wherein R³ in each occurrence is H or a substituent and Ar¹ is an unsubstituted or substituted C₆₋₂₀ arylene or C₅₋₂₀ heteroarylene group.

3. The compound according to claim 2 wherein Ar¹ is unsubstituted or substituted 1,2-phenylene.

4. The compound according to claim 2 wherein each R² is a group of formula (III).

5. A compound of formula (I): 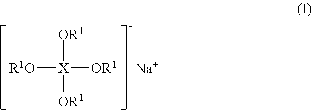
wherein X is Al or B; and
R¹ in each occurrence is independently a substituent and two R¹ groups may be linked to form a ring, with the proviso that at least one R¹ is a partially fluorinated linear or branched C₁₋₂₀ alkyl group in which one or more non-adjacent, non-terminal C atoms other than the C atom bound to O of X—O may be replaced with O and wherein the fluorinated C₁₋₂₀ alkyl group includes at least one group of formula —CF₂H or —CFH₂.

6. The compound according to claim 5 wherein each R¹ is a partially fluorinated linear or branched C₁₋₂₀ alkyl group in which one or more non-adjacent, non-terminal C atoms other than the C atom bound to O of X—O may be replaced with O and wherein the fluorinated C₁₋₂₀ alkyl group includes at least one group of formula —CF₂H or —CFH₂.

7. An electrolyte comprising a compound according to claim 1 and one or more solvents.

8. The electrolyte according to claim 7 wherein the one or more solvents include at least one of C₂₋₁₀ alkylene carbonates and di(C₁₋₁₀ alkyl) carbonates.

9. The electrolyte according to claim 7 wherein the electrolyte contains at least one mole of sodium cations per 10 moles of solvent.

10. The electrolyte according to claim 7 wherein the concentration of the compound of formula (II) is at least 0.25 moles/litre.

11. A battery comprising an anode, a cathode and an electrolyte according to claim 7 disposed between the anode and the cathode.

12. The battery according to claim 11 wherein the battery contains no more than 10 moles of total solvent per mole of Na+.

13. An electrolyte comprising a compound according to claim 5 and one or more solvents.

14. The electrolyte according to claim 13 wherein the one or more solvents include at least one of C₂₋₁₀ alkylene carbonates and di(C₁₋₁₀ alkyl) carbonates.

15. The electrolyte according to claim 13 wherein the electrolyte contains at least one mole of sodium cations per 10 moles of solvent.

16. The electrolyte according to claim 13 wherein the concentration of the compound of formula (I) is at least 0.25 moles/litre.

17. A battery comprising an anode, a cathode and an electrolyte according to claim 13 disposed between the anode and the cathode.

18. The battery according to claim 17 wherein the battery contains no more than 10 moles of total solvent per mole of Na+.
